(19) United States
(12) Patent Application Publication
(10) Pub. No.: US 2023/0129150 A1
(43) Pub. Date: Apr. 27, 2023

(54) RESPIRATOR FACE MASKS FOR PROTECTION FROM AIRBORNE PARTICLES

(71) Applicant: ENCELLIN, INC., San Francisco, CA (US)

(72) Inventors: Crystal NYITRAY, San Francisco, CA (US); Grace WEI, Tiburon, CA (US)

(21) Appl. No.: 17/914,004

(22) PCT Filed: Mar. 25, 2021
(86) PCT No.: PCT/US2021/024131

§ 371 Date: Sep. 23, 2022

Related U.S. Application Data
(60) Provisional application No. 63/000,081, filed on Mar. 26, 2020.

(51) Int. Cl. A61L 9/012; A41D 13/11; A61L 9/014; A62B 23/02; B01D 46/54; B01D 71/06; B01D 69/12; B01D 69/02

(52) U.S. Cl. CPC A61L 9/012; A41D 13/11; A61L 9/014; A62B 23/025; B01D 46/543; B01D 71/06; B01D 69/12; B01D 69/02; A61L 2209/14; A61L 2209/22; A62B 18/084

(57) ABSTRACT

The present disclosure provides a filtration media for filtering one or more airborne particles. The filtration media may comprise a membrane comprising one or more polymeric layers to filter the airborne particles. The filtration media may have high filtration efficiency and high breathability. Additionally, the present disclosure provides devices (e.g., face masks) comprising the filtration media which may be used to filter airborne particles (e.g., bacteria, viruses) and to protect the wearer (e.g., from infection).

### CROSS-REFERENCE

This application claims priority to U.S. Provisional Patent Application No. 63/000,081 filed on Mar. 26, 2020, which application is incorporated herein by reference in its entirety for all purposes.

### BACKGROUND

Breathable air may become contaminated with harmful substances such as particles, disease-causing microorganisms (e.g., bacteria, virus, fungi), chemicals, dust, fluids, soot, smoke, and other harmful airborne particles. During an ongoing threat of air contamination, such as rapid spread of an airborne disease-causing virus, there is a great need for respirator face masks that can effectively filter out the air contaminant and protect the wearer. Healthcare workers and other first responders often need to wear respirator face masks for extended periods of time. However, respirator face masks with high filtration efficiency often have breathability issues making it uncomfortable to wear for long periods of time. Additionally, during a global event, such as the COVID-19 pandemic, there may be critical supply shortages of respirator face masks.

### SUMMARY

There is a need for respirator face masks that have high filtration efficiency to effectively capture airborne particles (e.g., bacteria, viruses), and that have high breathability. There is also a need for respirator face masks that are easy and cost effective to manufacture.

Recognized herein are various shortcomings associated with the efficiency, breathability, and delta P performance of commercially-available filtration devices. The filtration media and filtration devices disclosed herein may comprise one or more layers or membranes with a pore size and/or pore structure that minimizes a passage of one or more particles through the layers or membranes while enhancing breathability. The filtration media disclosed herein may exhibit high filtration efficiency while minimizing a delta P (i.e., delta pressure or differential pressure) across the filtration media while the filtration media is in use (e.g., when a subject is breathing or inhaling one or more ambient air particles through the one or more layers or membranes of the filtration media). The filtration media and the filtration devices disclosed herein may provide users with an effective filtration barrier that is both comfortable and safe to use.

In one aspect, the present disclosure provides a filtration media for filtering one or more particles. The filtration media may comprise a membrane comprising one or more polymeric layers configured to filter one or more particles, wherein the membrane comprises a plurality of pores having an average diameter of at most about 175 μm (micrometers), wherein the membrane provides a filtration efficiency of at least about 65%, and wherein the membrane exhibits a delta P of at most about 5 mm H₂O/cm².

In some embodiments, the polymeric layer comprises a polymer selected from the group consisting of methacrylate polymer, polyethylene-imine, dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapetite, poly(lactic acid), polyhydroxyvalerte, polyhydroxybutyrate, polycaprolactone, polydiaxanone, polyanhydride, polycyanocrylate, poly(amino acid), poly(orthoester), polyester, collagen, gelatin, cellulose polymers, chitosan, alginate, polypropylene, polystyrene, Teflon, and polyethylene terephthalate (PET). In some embodiments, at least one of the one or more polymeric layers comprises an active agent configured to devitalize or deactivate one or more microorganisms or toxins associated with the one or more particles. In some embodiments, at least one of the one or more polymeric layers is positively or negatively charged to bind to the one or more particles. In some embodiments, at least one of the one or more polymeric layers is acidic. In some embodiments, the average diameter of the plurality of pores is from about 125 μm (micrometers) to about 175 μm. In some embodiments, the plurality of pores have an average connectivity diameter ranging from about 10 nanometers (nm) to about 200 micrometers (μm). In some embodiments, the one or more polymeric layers comprise a first polymeric layer with a first set of properties and a second polymeric layer with a second set of properties. In some embodiments, the first and second set of properties comprise a polymeric layer thickness, a pore size, a pore connectivity diameter, a pore density, or any combination thereof. In some embodiments, the first and second set of properties are the same. In some embodiments, the first and second set of properties are different. In some embodiments, at least one of the one or more polymeric layers is woven, spun, electrospun, blown, molded, or etched. In some embodiments, at least one of the one or more polymeric layers is die cut, sewn, molded, injection molded, cast, stamped, printed, or extruded. In some embodiments, at least one of the one or more polymeric layers is fabricated by extrusion, casting, or molding. In some embodiments, at least one of the one or more polymeric layers comprise a film or a scaffold. In some embodiments, the membrane comprises a single-use membrane. In some embodiments, the membrane comprises a reusable membrane. In some embodiments, the membrane has a filtration efficiency of at least about 70%. In some embodiments, the membrane has a filtration efficiency of at least about 75%. In some embodiments, the membrane has a filtration efficiency of at least about 80%. In some embodiments, the membrane has a filtration efficiency of at least about 85%. In some embodiments, the membrane has a filtration efficiency of at least about 90%. In some embodiments, the membrane has a filtration efficiency of at least about 95%. In some embodiments, the delta P of the membrane is from about 0.2 mm H₂O/cm² to about 5 mm H₂O/cm². In some embodiments, the size of the one or more particles is at least about 1 nanometer (nm), 10 nanometers, 100 nanometers, 1 micrometer (μm), 10 μm, 100 μm, 1 millimeter (mm), or more. In some embodiments, the size of the one or more particles is at most about 1 mm, 100 μm, 10 μm, 1 μm, 100 nm, 10 nm, 1 nm, or less. In some embodiments, the membrane comprising the plurality of pores has a density that is from about 0.1 mg/mm³ to about 0.2 mg/mm³. In some embodiments, the membrane comprising the plurality of pores has a density that is from about 0.01 mg/mm³ to about 1 mg/mm³. In some embodiments, the membrane comprising the plurality of pores has a pore density that is from about 10,000 pores/mm² to about 100,000 pores/mm². In some embodiments, the membrane comprising the plurality of pores has a pore density of at least about 100,000 pores/mm².

In another aspect, the present disclosure provides a device for filtering one or more particles. The device may comprise a filtration media and at least one securement member for securing the filtration media to a subject or an object. In some embodiments, the device may further comprise an additional layer comprising a condensation wicking or absorbent material. In some embodiments, the additional layer comprises one or more natural fibers or natural-like fibers. In some embodiments, the device comprises a component configured to receive the filtration media. In some embodiments, the filtration media is swappable or switchable after one or more uses. In some embodiments, the device comprises a face mask (e.g., a respirator face mask).

In another aspect, the present disclosure provides a mask for filtering one or more airborne particles, comprising: a membrane comprising one or more polymeric layers configured to filter one or more particles, wherein the one or more polymeric layers comprise polycaprolactone, wherein the membrane comprises a plurality of pores; and at least one securement member for securing the membrane to a subject or an object. In some embodiments, the plurality of pores have an average diameter that is from about 125 μm (micrometers) to about 175 μm. In some embodiments, the mask has a filtration efficiency of at least about 90%. In some embodiments, the mask exhibits a delta P that is from about 0.2 mm H₂O/cm² to about 5 mm H₂O/cm².

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A, 1B, 1C, and 1D schematically illustrate various exemplary embodiments of a filtration media comprising one or more layers as provided herein.

FIGS. 2A and 2B schematically illustrate a structure of an exemplary membrane of a filtration media as imaged using a scanning electron microscope.

### DETAILED DESCRIPTION

The present disclosure provides various filtration media and filtration devices for filtering one or more particles within a fluid medium (e.g., a liquid or a gas). The filtration media and filtration devices disclosed herein may comprise one or more layers or membranes with a pore size and/or pore structure that minimizes a passage of one or more particles through the layers or membranes while enhancing breathability. The filtration media disclosed herein may exhibit high filtration efficiency while minimizing a delta P (i.e., delta pressure or differential pressure) across the filtration media while the filtration media is in use (e.g., when a subject is breathing or inhaling one or more ambient air particles through the one or more layers or membranes of the filtration media). The filtration media and the filtration devices disclosed herein may provide users with an effective filtration barrier that is both comfortable and safe to use.

In addition to the benefits and advantages above, the filtration media and filtration devices of the present disclosure exhibit certain other highly desirable properties such as biodegradability due to a chemical structure of the layers or membranes of the filtration media. In some cases, biodegradability may be determined based on one or more industry test standards relating to biodegradation (e.g., American Society for Testing and Materials (ASTM) D5338, D6400, D6868, etc.) In other cases, biodegradability may be determined based on an amount of degradation of a portion of the filtration media within a predetermined time frame (under controlled conditions and/or in the presence of one or more microorganisms such as bacteria or fungi). The amount of degradation may be at least about 1%, at least about 5%, at least about 10%, at least about 20%, at least about 30%, at least about 40%, at least about 50%, at least about 60%, at least about 70%, at least about 80%, at least about 90%, or more of the filtration media by weight. The predetermined time frame may be at most about 5 years, at most about 4 years, at most about 3 years, at most about 2 years, at most about 1 year, at most about 9 months, at most about 6 months, at most about 3 months, at most about 1 month, at most about 30 days, at most about 20 days, at most about 10 days, at most about 5 days, at most about 1 day, or less. The filtration media disclosed herein may be considered biodegradable if the filtration media is capable of undergoing any amount of degradation (by weight) within the predetermined time frames disclosed herein. Further, the filtration media of the present disclosure may comprise a crystal structure that enhances the reusability of the filtration media and the one or more layers or membranes of the filtration media. In some cases, the crystal structure may permit the one or more layers or membranes to be melted and remade, refurbished, refashioned, reshaped, and/or reconditioned (e.g., for reuse or for use in another filtration device).

Filtration Media

In an aspect, the present disclosure provides a filtration media comprising one or more membranes for filtering one or more particles. As used herein, a “membrane” may comprise a thin article of material (e.g., in the form of a sheet, annulus, hollow hemisphere, etc.) that is permeable to certain substances or particles. The membrane may also be non-permeable or semi-permeable to various other substances or particles.

In some cases, the filtration media may comprise a plurality of membranes. The membranes may be coupled together (e.g., using a fastening mechanism or an adhesive). Alternatively, the membranes may not or need not be coupled together.

Particles

In some cases, the one or more particles may comprise one or more airborne particles that may be transported through or suspended in ambient air or a gas. In other cases, the one or more particles may be suspended in a liquid (e.g., one or more particles or molecules suspended in a solution). The one or more particles may have a particle size corresponding to a dimension (e.g., a length, a width, a height, a circumference, a diameter, a perimeter, etc.) of the one or more particles. The particle size may be at least about 1 nanometer (nm), at least about 10 nanometers, at least about 100 nanometers, at least about 1 micrometer (μm), at least about 10 μm, at least about 100 μm, at least about 1 millimeter (mm), or more. In some embodiments, the particle size may be at most about 1 mm, at most about 100 μm, at most about 10 μm, at most about 1 μm, at most about 100 nm, at most about 10 nm, at most about 1 nm, or less.

In some cases, the one or more particles may comprise an irritant, an allergen, a microbe, and/or a toxic or harmful substance. In other cases, the one or more particles may comprise a particle that can cause one or more health conditions or ailments in a subject (e.g., mucosa irritation, rash, severe allergic reactions, fatigue, headache, nausea, depression, and/or an increased risk of throat or lung cancer).

In some cases, the one or more particles may comprise a volatile organic compound (VOC). The VOC may comprise, for example, gaseous formaldehyde, benzene, toluene, styrene, acetone, para-dichlorobenzene, chloroform, tetrachloroethylene, acrylic acid esters, aliphatic ketones, alcohol, volatile sulfur complexes, and/or ammonia gas.

In some cases, the one or more particles may comprise one or more infection or disease-causing particles. The infection or disease-causing particles may comprise one or more viruses. The one or more viruses may comprise, for example, adenoviruses, herpesviruses, poxviruses, parvoviruses, papovaviruses, hepadnaviruses, orthomyxoviruses, paramyxoviruses, coronaviruses, picornaviruses, reoviruses, togaviruses, flaviviruses, arenaviruses, rhabdoviruses, and/or retroviruses.

In some cases, the one or more infection or disease-causing particles may comprise bacteria or one or more bacterial strains. The one or more bacterial strains may comprise, for example, Staphylococcus aureus, Streptococcus pneumoniae, Streptococcus pyogenes, Neisseria gonorrhoeae, Neisseria meningitides, Corynebacteria diphtheriae, Bacillus anthracis, Listeria monocytogenes, Escherichia coli, Salmonella typhimurium, Salmonella typhae, Shigella, Campylobacter, Vibrio cholerae, Yersinia pestis, Pasteurella, Pseudomonas aeruginosa, Brucella Haemophilus, influenzae, Legionella, Bordetella, Clostridium tetani, Clostridium botulinum, Clostridium perfringens, Treponema pallidum, Borrelia burgdorferi, Leptospira interrogans, Mycobacterium tuberculosis, Mycobacterium leprae, Mycobacterium avium, Rickettsia prowazekii, Chlamydia trachomatis, and/or Mycoplasma pneumoniae.

Filtration Device

In some cases, the filtration media may be provided separately from a filtration device. In other cases, the filtration media may be integrated with the filtration device. As used herein, a filtration device may comprise any device or apparatus that is usable to prevent a passage of one or more undesirable particles into a subject's body or a system. The system may comprise any mechanical or non-mechanical system with one or more components that can perform a filtration of certain desirable or undesirable particles from a transfer medium (e.g., a gas or a liquid). The one or more components may be in fluid communication with the transfer medium comprising the desirable or undesirable particles. The system may or may not require the filtration of said desirable or undesirable particles in order to function properly.

In some cases, the filtration device may comprise a mask (e.g., a respirator mask). The respirator mask may be configured for placement over a portion of a subject's face. The portion of the subject's face may include, for example, a nose and/or a mouth portion of the subject. In some cases, the filtration device may comprise a device that is placed or secured on or near one or more airways of the subject (e.g., a nasal passage or a mouth of the subject). In some cases, the filtration device may comprise personal protective equipment.

In some cases, the filtration device may comprise any type of filter used for filtering air input into a pneumatic system, or fluid input into a fluidic system. The pneumatic system may comprise, for example, an air filtration system for buildings and/or vehicles that is intended to improve the safety and/or quality of air. In some cases, the pneumatic system may comprise various heating, ventilating and air conditioning (HVAC) systems. The fluidic system may comprise, for example, a hydraulic system or a fluid filtration system associated with a chemical plant (e.g., a water treatment facility) or the chemical processing of one or more fluids, materials, or substances.

In some cases, the filtration media may be coupled to and/or releasably attached to the filtration device. In some cases, the filtration media may be insertable into a component or a portion of a body of the filtration device. The filtration media may be removable from the filtration device and/or swappable after one or more uses. In other cases, the filtration media may not be reusable or removable from the filtration device after one or more uses.

Respirator Mask

In one aspect, provided herein is a respirator face mask to protect a subject from one or more particles and/or microorganisms such as disease-causing viruses. In some embodiments, the respirator face mask may comprise a filtration media that comprises a membrane with one or more polymeric layers to filter airborne particles and microorganisms.

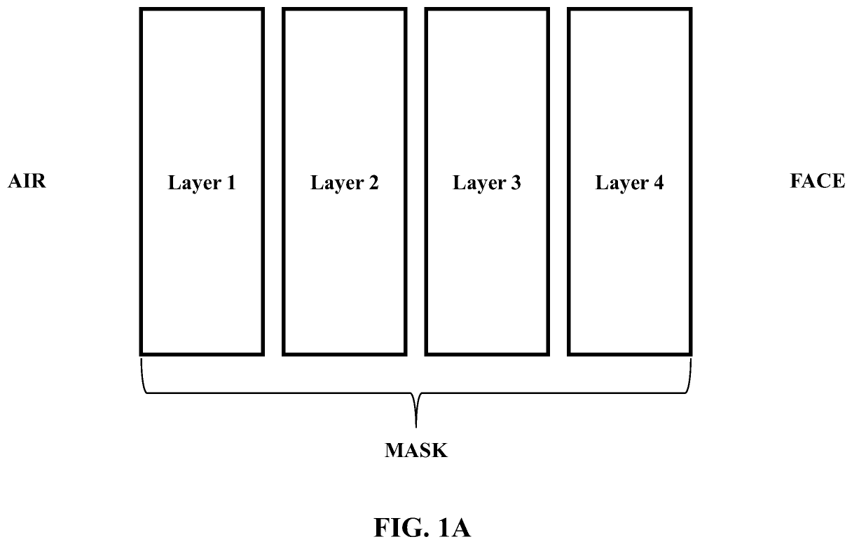

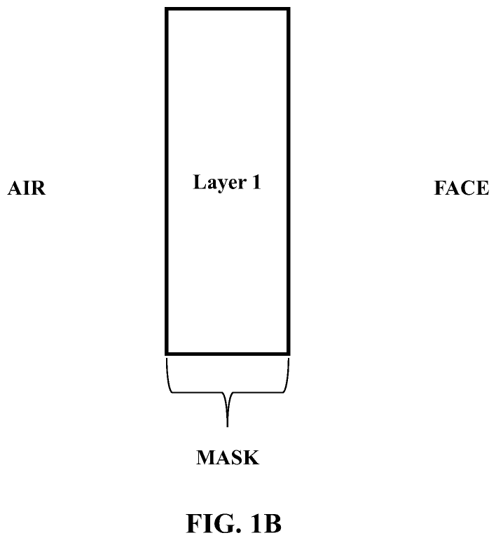

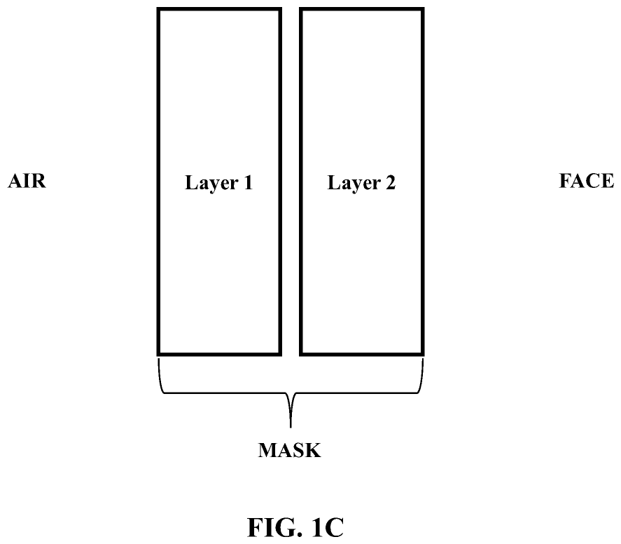

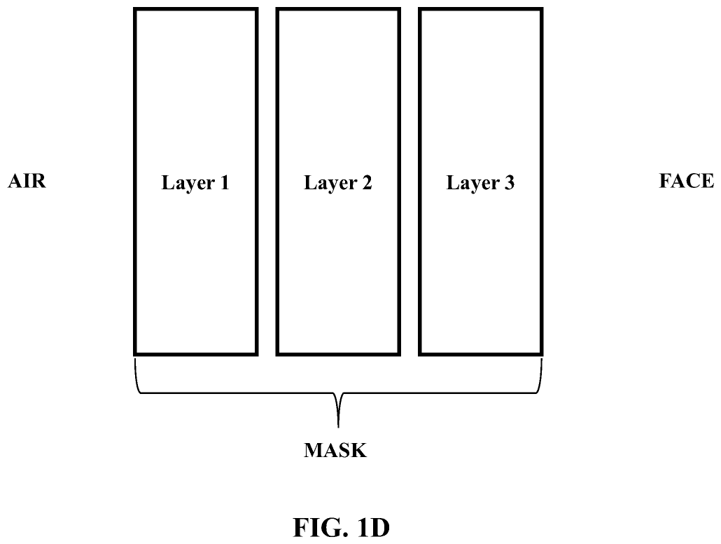

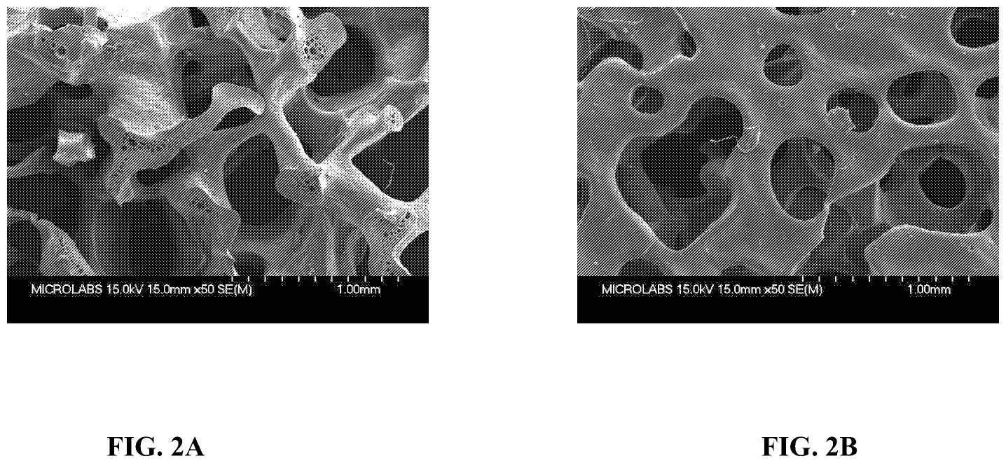

In some embodiments, the filtration media comprises a first layer that may be exposed to external air to shield the subject wearing the respirator face mask from airborne pathogens, for example, as shown in FIG. 1A. In some cases, the first layer comprises a synthetic polymer. In some cases, the first layer comprises a non-synthetic polymer. In other cases, the first layer comprises a synthetic polymer and a non-synthetic polymer. The membrane of the filtration media may comprise a structure, for example, as shown in FIGS. 2A and 2B. Such a structure may exhibit favorable and advantageous properties such as high filtration efficiency and breathability, as indicated by the experimental test data shown below.

In some embodiments, the filtration device may comprise a filtration media and at least one securement member for securing the filtration media to a subject or an object. The securement member may comprise, for example, a loop, a strap, or a band. In some cases, the device may further comprise an additional layer comprising a condensation wicking or absorbent material. In some embodiments, the additional layer may comprise one or more natural fibers or natural-like fibers.

In some embodiments, the device comprises a component configured to receive the filtration media. The component may comprise a structural component of the device with an opening and an inner volume or space configured to receive or hold the filtration media. In some embodiments, the filtration media is swappable or switchable after one or more uses.

In some embodiments, the device comprises a face mask (e.g., a respirator face mask). The face mask may comprise a membrane comprising one or more polymeric layers to filter one or more particles. The one or more polymeric layers may comprise polycaprolactone. In some cases, the membrane may comprise a plurality of pores arranged on at least a portion of the membrane. In some embodiments, the plurality of pores have an average diameter that is from about 125 μm (micrometers) to about 175 μm. In some cases, the face mask may comprise at least one securement member for securing the membrane to a subject or an object.

In some cases, the mask may have a filtration efficiency of at least about 65%. In some cases, the mask may have a filtration efficiency of at least about 70%. In some cases, the mask may have a filtration efficiency of at least about 80%. In some cases, the mask may have a filtration efficiency of at least about 90%. In some cases, the mask may exhibit a delta P that is from about 0.2 mm H₂O/cm² to about 5 mm H₂O/cm².

Layer Composition and Properties

In some cases, the one or more layers of the membrane or the filtration media can comprise a polymer or a polymeric material. The polymer or polymeric material may comprise, for example, methacrylate polymer, polyethylene-imine and dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapetite, poly(lactic acid), polyhydroxyvalerte and copolymers, polyhydroxybutyrate and copolymers, polycaprolactone, polydiaxanone, polyanhydrides, polycyanocrylates, poly(amino acids), poly(orthoesters), polyesters, collagen, gelatin, cellulose polymers, chitosans, alginates, polycaprolactone (PCL), polyvinylidene fluoride (PVDF), polytetrafluoroethylene (PTFE), polyethylene (PE), methacrylate polymer, polyethyleneimine, polyethyleneimine-dextran sulfate, poly(vinylsiloxane), laminin, Teflon, polyethylene terephthalate (PET), polypropylene (e.g., electrospun polypropylene), or a combination thereof. In some cases, the polymer is poly(caprolactone)(PCL). In some cases, the layers of the membrane or the filtration media may comprise a same material. In other cases, the layers of the membrane or the filtration media may comprise different materials.

The first layer of the respirator face mask may be a film. The first layer may be a scaffold. The first layer may be woven. The first layer may be spun (e.g., electrospun). The first layer may be blown. The first layer may be molded. The first layer may be etched. In some case, the first layer may be fabricated by extrusion, casting, or molding. In some embodiments, the first layer may be die cut, sewn, molded, injection molded, cast, stamped, printed, or extruded.

The first layer can be acidic in nature. The first layer may comprise an acidic material. Alternatively, the first layer may comprise one or more acidic molecules or compounds. The acidity of the first layer can depolarize one or more pathogens such as bacteria, fungi, and virus that come into contact with the first layer. The pH of the first layer may range from about 5 to about 6. The pH of the first layer may be about 5. The pH of the first layer may be about 5.5. The pH of the first layer may be about 6. The pH of the first layer may be less than 6. In some embodiments, the first layer comprises an acid such as citric acid to provide its acidic characteristic. In some cases, the final concentration of the acid in the first layer is about 2% w/w. In some cases, the final concentration of the acid in the first layer is less than 2% w/w. In other cases, the final concentration of the acid in the first layer is greater than 2% w/w.

In certain embodiments, the first layer may comprise an active agent. The active agent can be a biocidal iodinated resin. In alternative embodiments, the active agent comprises triclosan (a polychloro phenoxy phenol), diatomic halogen, silver, copper, zeolyte with an antimicrobial attached thereto, halogenated resin, or other agent capable of devitalizing/deactivating microorganisms/toxins such as activated carbon, phenol (carbolic acid) compounds, terpenes, other metals, certain acids and bases, and other chemical compounds.

The first layer may be positively charged or negatively charged to bind to airborne microorganisms such as bacteria, fungi, and virus. The first layer may comprise one or more metal ions, one or more electrophiles, one or more nucleophiles, or a combination thereof. The one or more metal ions may be copper, zinc, or a combination thereof. In some cases, the concentration of the one or more metal ions, one or more electrophiles, one or more nucleophiles, or a combination thereof, may be about 1.6% w/w, less than 1.6% w/w, or greater than 1.6% w/w. In some cases, the first layer (and/or any other layer of the membrane/filtration media) may comprise one or more positively or negatively charged ions/particles distributed, embedded, or incorporated into the layer or a portion of the layer. In some cases, the first layer (or any portion or region of the first layer) may be positively or negatively charged by doping. The first layer (and/or any other layer of the membrane/filtration media) may be doped by adding chemical reactants to induce one or more oxidization or reduction reactions that cause one or more electrons to be pushed into one or more conducting orbitals within the layer of the membrane/filtration media. In some embodiments, doping may comprise chemical doping, which involves exposing a material to an oxidant such as iodine or bromine. Alternatively, the material can be exposed to a reductant, typically an alkali metal. In other embodiments, doping may comprise electrochemical doping, which involves suspending a polymer-coated, working electrode in an electrolyte solution in which the polymer is insoluble, along with separate counter and reference electrodes. An electric potential difference can be created between the electrodes that induces a charge and causes the appropriate counter ion from the electrolyte to enter the polymer in the form of electron addition (i.e., n-doping) or removal (i.e., p-doping).

In some cases, one or more dopants may be used to produce the positively or negatively charged layer. The one or more dopants may comprise, for example, boron, aluminum, gallium, indium, phosphorus, arsenic, antimony, bismuth, lithium, germanium, tellurium, sulfur, selenium, tin, silicon, beryllium, zinc, chromium, magnesium, carbon, chlorine, sodium, iodine, fluorine, or any other suitable element usable to induce a positive or negative charge in the layer (or a portion thereof).

In some embodiments, the thickness of the first layer is about 1 mm to about 2 mm. In some embodiments, the thickness of the first layer is at least about 1 mm. In some embodiments, the thickness of the first layer is at most about 2 mm. In some embodiments, the thickness of the first layer is about 1 mm to about 1.5 mm, about 1 mm to about 2 mm, or about 1.5 mm to about 2 mm. In some embodiments, the thickness of the first layer is about 1 mm, about 1.5 mm, or about 2 mm.

In some embodiments, the average diameter of the pores of the first layer is about 50 μm to about 500 μm. In some embodiments, the average diameter of the pores of the first layer is at least about 50 μm. In some embodiments, the average diameter of the pores of the first layer is at most about 500 μm. In some embodiments, the average diameter of the pores of the first layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 75 μm to about 100 μm, about 75 μm to about 150 μm, about 75 μm to about 200 μm, about 75 μm to about 250 μm, about 75 μm to about 300 μm, about 75 μm to about 350 μm, about 75 μm to about 400 μm, about 75 μm to about 450 μm, about 75 μm to about 500 μm, about 100 μm to about 150 μm, about 100 μm to about 200 μm, about 100 m to about 250 μm, about 100 μm to about 300 μm, about 100 μm to about 350 μm, about 100 μm to about 400 μm, about 100 μm to about 450 μm, about 100 μm to about 500 μm, about 150 μm to about 200 μm, about 150 μm to about 250 μm, about 150 μm to about 300 μm, about 150 μm to about 350 μm, about 150 μm to about 400 μm, about 150 μm to about 450 μm, about 150 μm to about 500 μm, about 200 μm to about 250 μm, about 200 μm to about 300 μm, about 200 μm to about 350 μm, about 200 μm to about 400 μm, about 200 μm to about 450 μm, about 200 μm to about 500 μm, about 250 μm to about 300 μm, about 250 μm to about 350 μm, about 250 μm to about 400 μm, about 250 μm to about 450 μm, about 250 μm to about 500 μm, about 300 μm to about 350 μm, about 300 μm to about 400 μm, about 300 μm to about 450 μm, about 300 μm to about 500 μm, about 350 μm to about 400 μm, about 350 μm to about 450 μm, about 350 μm to about 500 μm, about 400 μm to about 450 μm, about 400 μm to about 500 m, or about 450 μm to about 500 μm. In some embodiments, the average diameter of the pores of the first layer is about 50 μm, about 75 μm, about 100 μm, about 150 μm, about 200 μm, about 250 μm, about 300 μm, about 350 μm, about 400 μm, about 450 μm, or about 500 μm.

In some embodiments, the average diameter of the pores of the first layer is about 500 μm to about 1,000 μm. In some embodiments, the average diameter of the pores of the first layer is at least about 500 μm. In some embodiments, the average diameter of the pores of the first layer is at most about 1,000 μm. In some embodiments, the average diameter of the pores of the first layer is about 500 μm to about 550 μm, about 500 μm to about 600 μm, about 500 μm to about 650 μm, about 500 μm to about 700 μm, about 500 μm to about 750 μm, about 500 μm to about 800 μm, about 500 μm to about 850 μm, about 500 μm to about 900 μm, about 500 μm to about 950 μm, about 500 μm to about 1,000 μm, about 550 μm to about 600 μm, about 550 μm to about 650 μm, about 550 μm to about 700 μm, about 550 μm to about 750 μm, about 550 μm to about 800 μm, about 550 μm to about 850 μm, about 550 μm to about 900 μm, about 550 μm to about 950 μm, about 550 μm to about 1,000 μm, about 600 μm to about 650 μm, about 600 μm to about 700 μm, about 600 μm to about 750 μm, about 600 μm to about 800 μm, about 600 μm to about 850 μm, about 600 μm to about 900 μm, about 600 μm to about 950 μm, about 600 μm to about 1,000 μm, about 650 μm to about 700 μm, about 650 μm to about 750 μm, about 650 nm to about 800 μm, about 650 μm to about 850 μm, about 650 μm to about 900 μm, about 650 nm to about 950 μm, about 650 μm to about 1,000 μm, about 700 μm to about 750 μm, about 700 μm to about 800 μm, about 700 μm to about 850 μm, about 700 μm to about 900 μm, about 700 μm to about 950 μm, about 700 μm to about 1,000 μm, about 750 μm to about 800 μm, about 750 μm to about 850 μm, about 750 μm to about 900 μm, about 750 μm to about 950 μm, about 750 μm to about 1,000 μm, about 800 μm to about 850 μm, about 800 μm to about 900 μm, about 800 μm to about 950 μm, about 800 μm to about 1,000 μm, about 850 μm to about 900 μm, about 850 μm to about 950 μm, about 850 μm to about 1,000 μm, about 900 μm to about 950 μm, about 900 μm to about 1,000 μm, or about 950 μm to about 1,000 μm. In some embodiments, the average diameter of the pores of the first layer is about 500 μm, about 550 μm, about 600 μm, about 650 μm, about 700 μm, about 750 μm, about 800 μm, about 850 μm, about 900 μm, about 950 μm, or about 1,000 μm.

In some embodiments, the thickness of the first layer is about 1 mm, and the average diameter of the pores of the first layer is about 100 μm to about 1,000 μm. In some embodiments, the thickness of the first layer is about 1 mm, and the average diameter of the pores of the first layer is at least about 100 μm. In some embodiments, the thickness of the first layer is about 1 mm, and the average diameter of the pores of the first layer is at most about 1,000 μm. In some embodiments, the thickness of the first layer is about 1 mm, and the average diameter of the pores of the first layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the first layer is about 1 mm, and the average diameter of the pores of the first layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the first layer is about 1.5 mm, and the average diameter of the pores of the first layer is about 100 μm to about 1,000 μm. In some embodiments, the thickness of the first layer is about 1.5 mm, and the average diameter of the pores of the first layer is at least about 100 μm. In some embodiments, the thickness of the first layer is about 1.5 mm, and the average diameter of the pores of the first layer is at most about 1,000 μm. In some embodiments, the thickness of the first layer is about 1.5 mm, and the average diameter of the pores of the first layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the first layer is about 1.5 mm, and the average diameter of the pores of the first layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the first layer is about 2 mm, and the average diameter of the pores of the first layer is about 100 μm to about 1,000 μm. In some embodiments, the thickness of the first layer is about 2 mm, and the average diameter of the pores of the first layer is at least about 100 μm. In some embodiments, the thickness of the first layer is about 2 mm, and the average diameter of the pores of the first layer is at most about 1,000 μm. In some embodiments, the thickness of the first layer is about 2 mm, and the average diameter of the pores of the first layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the first layer is about 2 mm, and the average diameter of the pores of the first layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the first layer is about 1 mm, and the average diameter of the pores of the first layer is about 50 μm to about 355 μm. In some embodiments, the thickness of the first layer is about 1 mm, and the average diameter of the pores of the first layer is at least about 50 μm. In some embodiments, the thickness of the first layer is about 1 mm, and the average diameter of the pores of the first layer is at most about 355 μm. In some embodiments, the thickness of the first layer is about 1 mm, and the average diameter of the pores of the first layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the first layer is about 1 mm, and the average diameter of the pores of the first layer is about 50 μm, about 75 μm, about 100 μm, about 150 μm, about 200 μm, about 250 μm, about 300 μm, or about 355 μm.

In some embodiments, the thickness of the first layer is about 1.5 mm, and the average diameter of the pores of the first layer is about 50 μm to about 355 μm. In some embodiments, the thickness of the first layer is about 1.5 mm, and the average diameter of the pores of the first layer is at least about 50 μm. In some embodiments, the thickness of the first layer is about 1.5 mm, and the average diameter of the pores of the first layer is at most about 355 μm. In some embodiments, the thickness of the first layer is about 1.5 mm, and the average diameter of the pores of the first layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the first layer is about 1.5 mm, and the average diameter of the pores of the first layer is about 50 μm, about 75 μm, about 100 μm, about 150 μm, about 200 μm, about 250 μm, about 300 μm, or about 355 μm.

In some embodiments, the thickness of the first layer is about 2 mm, and the average diameter of the pores of the first layer is about 50 μm to about 355 μm. In some embodiments, the thickness of the first layer is about 2 mm, and the average diameter of the pores of the first layer is at least about 50 μm. In some embodiments, the thickness of the first layer is about 2 mm, and the average diameter of the pores of the first layer is at most about 355 μm. In some embodiments, the thickness of the first layer is about 2 mm, and the average diameter of the pores of the first layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the first layer is about 2 mm, and the average diameter of the pores of the first layer is about 50 μm, about 75 μm, about 100 μm, about 150 μm, about 200 μm, about 250 μm, about 300 μm, or about 355 μm.

In some cases, the polymer of the first layer may be nanoporous, wherein the average diameter of the pores ranges from about 30 nm to about 100 nm, such as, for example, about 30 to about 75 nm, about 30 to about 50 nm, about 40 to about 100 nm, from about 40 to about 75 nm, from about 40 to about 50 nm, from about 50 to about 100 nm, or from about 50 to about 75 nm, e.g., about 30 nm, about 40 nm, about 50 nm, about 60 nm, or about 80 nm.

In other cases, the polymer of the first layer may be microporous, wherein the average diameter of the pores ranges from about 1 μm to about 3 μm (e.g., about 2 μm±about 0.5 μm diameter) with a connectivity diameter ranging from about 900 nm to about 100 nm, such as, from about 900 nm to about 150 nm, from about 900 nm to about 200 nm, from about 700 nm to about 100 nm, from about 700 nm to about 150 nm, from about 700 nm to about 200 nm, from about 500 nm to about 100 nm, from about 500 nm to about 150 nm, from about 300 nm to about 100 nm, from about 300 nm to about 150 nm, or from about 300 nm to about 200 nm. In some cases, the average diameter of the pores may range from about 100 μm to about 200 μm. For example, the average diameter of the pores may be at least about 100 μm, at least about 105 μm, at least about 110 μm, at least about 115 μm, at least about 120 μm, at least about 125 μm, at least about 130 μm, at least about 135 μm, at least about 140 μm, at least about 145 μm, at least about 150 μm, at least about 155 μm, at least about 160 μm, at least about 165 μm, at least about 170 μm, at least about 175 μm, at least about 180 μm, at least about 185 μm, at least about 190 μm, at least about 195 μm, at least about 200 μm, or more. In some cases, the average diameter of the pores may be at most about 200 μm, at most about 195 μm, at most about 190 μm, at most about 185 μm, at most about 180 μm, at most about 175 μm, at most about 170 μm, at most about 165 μm, at most about 160 μm, at most about 155 μm, at most about 150 μm, at most about 145 μm, at most about 140 μm, at most about 135 μm, at most about 130 μm, at most about 125 μm, at most about 120 μm, at most about 115 μm, at most about 110 μm, at most about 105 μm, at most about 100 μm, or less.

In some cases, the polymer of the first layer may have an average connectivity diameter ranging from about 10 nm to about 2 μm. For example, the plurality of pores present in the first layer may have an average connectivity diameter ranging from about 20 nm to about 300 nm, from about 30 nm to about 300 nm, from about 10 nm to about 200 nm, from about 20 nm to about 200 nm, from about 30 nm to about 200 nm, from about 10 nm to about 100 nm, from about 20 nm to about 100 nm, from about 30 nm to about 100 nm, from about 30 nm to about 50 nm, from about 30 nm to about 40 nm, from about 20 nm to about 250 nm, from about 20 nm to about 150 nm, from about 25 to about 150 nm, from about 200 nm to about 500 nm, from about 350 nm to about 500 nm, from about 500 nm to about 1.5 μm, or from about 1.0 μm to about 2.0 μm. In some cases, the plurality of pores in the first layer may have an average connectivity diameter of about 10 nm, about 20 nm, about 30 nm, about 40 nm, about 50 nm, about 60 nm, about 70 nm, about 80 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 550 nm, about 600 nm, about 650 nm, about 700 nm, about 750 nm, about 800 nm, about 850 nm, about 900 nm, about 950 nm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, or about 2.0 μm. In one aspect, the average connectivity diameter may be about 200 nm. In another aspect, the average connectivity diameter may be about 2.0 μm.

In some embodiments, the respirator face mask may optionally comprise a second layer, for example, as shown in FIG. 1A. The second layer comprises a polymer. The polymer of the second layer may be a synthetic polymer. In some cases, the polymer of the second layer may be a non-synthetic polymer. In other cases, polymer of the second layer may comprise a synthetic polymer and a non-synthetic polymer.

In some cases, the polymer of the second layer can comprise methacrylate polymer, polyethylene-imine and dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapetite, poly(lactic acid), polyhydroxyvalerte and copolymers, polyhydroxybutyrate and copolymers, polycaprolactone, polydiaxanone, polyanhydrides, polycyanocrylates, poly(amino acids), poly(orthoesters), polyesters, collagen, gelatin, cellulose polymers, chitosans, alginates, polycaprolactone (PCL), polyvinylidene fluoride (PVDF), polytetrafluoroethylene (PTFE), polyethylene (PE), methacrylate polymer, polyethyleneimine, polyethyleneimine-dextran sulfate, poly(vinylsiloxane), laminin, Teflon, polyethylene terephthalate (PET), or a combination thereof. In some cases, the polymer is poly(caprolactone)(PCL).

The second layer of the respirator face mask may be a scaffold. The second layer may be woven. The second layer may be spun (e.g., electrospun). The second layer may be blown. The second layer may be molded. The second layer may be etched. In some embodiments, at least one of the one or more polymeric layers is fabricated by extrusion, casting, or molding. In some embodiments, the second layer may be die cut, sewn, molded, injection molded, cast, stamped, printed, or extruded.

The second layer may be positively charged or negatively charge to bind to airborne microorganisms such as bacteria, fungi and virus. The second layer may comprise one or more metal ions, one or more electrophiles, one or more nucleophiles, or a combination thereof. The one or more metal ions may be copper, zinc, or a combination thereof.

In some embodiments, the thickness of the second layer is about 1 mm to about 2 mm. In some embodiments, the thickness of the second layer is at least about 1 mm. In some embodiments, the thickness of the second layer is at most about 2 mm. In some embodiments, the thickness of the second layer is about 1 mm to about 1.5 mm, about 1 mm to about 2 mm, or about 1.5 mm to about 2 mm. In some embodiments, the thickness of the second layer is about 1 mm, about 1.5 mm, or about 2 mm.

In some embodiments, the average diameter of the pores of the second layer is about 50 μm to about 500 μm. In some embodiments, the average diameter of the pores of the second layer is at least about 50 μm. In some embodiments, the average diameter of the pores of the second layer is at most about 500 μm. In some embodiments, the average diameter of the pores of the second layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 75 μm to about 100 μm, about 75 μm to about 150 μm, about 75 μm to about 200 μm, about 75 μm to about 250 μm, about 75 μm to about 300 μm, about 75 μm to about 350 μm, about 75 μm to about 400 μm, about 75 μm to about 450 μm, about 75 μm to about 500 μm, about 100 μm to about 150 μm, about 100 μm to about 200 μm, about 100 μm to about 250 μm, about 100 μm to about 300 μm, about 100 μm to about 350 μm, about 100 μm to about 400 μm, about 100 μm to about 450 μm, about 100 μm to about 500 μm, about 150 μm to about 200 μm, about 150 μm to about 250 μm, about 150 μm to about 300 μm, about 150 μm to about 350 μm, about 150 μm to about 400 μm, about 150 μm to about 450 μm, about 150 μm to about 500 μm, about 200 μm to about 250 μm, about 200 μm to about 300 μm, about 200 μm to about 350 μm, about 200 μm to about 400 μm, about 200 μm to about 450 μm, about 200 μm to about 500 μm, about 250 μm to about 300 μm, about 250 μm to about 350 μm, about 250 μm to about 400 μm, about 250 μm to about 450 μm, about 250 μm to about 500 μm, about 300 μm to about 350 μm, about 300 μm to about 400 μm, about 300 μm to about 450 μm, about 300 μm to about 500 μm, about 350 μm to about 400 μm, about 350 μm to about 450 μm, about 350 μm to about 500 μm, about 400 μm to about 450 μm, about 400 μm to about 500 μm, or about 450 μm to about 500 μm. In some embodiments, the average diameter of the pores of the second layer is about 50 μm, about 75 μm, about 100 μm, about 150 μm, about 200 μm, about 250 μm, about 300 μm, about 350 μm, about 400 μm, about 450 μm, or about 500 nm.

In some embodiments, the average diameter of the pores of the second layer is about 500 nm to about 1,000 μm. In some embodiments, the average diameter of the pores of the second layer is at least about 500 μm. In some embodiments, the average diameter of the pores of the second layer is at most about 1,000 μm. In some embodiments, the average diameter of the pores of the second layer is about 500 μm to about 550 μm, about 500 μm to about 600 μm, about 500 nm to about 650 μm, about 500 μm to about 700 μm, about 500 μm to about 750 μm, about 500 nm to about 800 μm, about 500 μm to about 850 μm, about 500 μm to about 900 μm, about 500 nm to about 950 μm, about 500 μm to about 1,000 μm, about 550 μm to about 600 μm, about 550 μm to about 650 μm, about 550 μm to about 700 μm, about 550 μm to about 750 μm, about 550 μm to about 800 μm, about 550 μm to about 850 μm, about 550 μm to about 900 μm, about 550 μm to about 950 μm, about 550 μm to about 1,000 μm, about 600 μm to about 650 μm, about 600 μm to about 700 μm, about 600 μm to about 750 μm, about 600 μm to about 800 μm, about 600 μm to about 850 μm, about 600 μm to about 900 μm, about 600 μm to about 950 μm, about 600 μm to about 1,000 μm, about 650 μm to about 700 μm, about 650 μm to about 750 nm, about 650 μm to about 800 μm, about 650 μm to about 850 μm, about 650 μm to about 900 nm, about 650 μm to about 950 μm, about 650 μm to about 1,000 μm, about 700 μm to about 750 μm, about 700 μm to about 800 μm, about 700 μm to about 850 μm, about 700 μm to about 900 μm, about 700 μm to about 950 μm, about 700 μm to about 1,000 μm, about 750 μm to about 800 μm, about 750 μm to about 850 μm, about 750 μm to about 900 μm, about 750 μm to about 950 μm, about 750 μm to about 1,000 μm, about 800 μm to about 850 μm, about 800 μm to about 900 μm, about 800 μm to about 950 μm, about 800 μm to about 1,000 μm, about 850 nm to about 900 μm, about 850 μm to about 950 μm, about 850 μm to about 1,000 μm, about 900 μm to about 950 μm, about 900 μm to about 1,000 μm, or about 950 μm to about 1,000 μm. In some embodiments, the average diameter of the pores of the second layer is about 500 μm, about 550 μm, about 600 μm, about 650 μm, about 700 μm, about 750 μm, about 800 μm, about 850 μm, about 900 μm, about 950 μm, or about 1,000 μm.

In some embodiments, the thickness of the second layer is about 1 mm, and the average diameter of the pores of the second layer is about 100 μm to about 1,000 μm. In some embodiments, the thickness of the second layer is about 1 mm, and the average diameter of the pores of the second layer is at least about 100 μm. In some embodiments, the thickness of the second layer is about 1 mm, and the average diameter of the pores of the second layer is at most about 1,000 μm. In some embodiments, the thickness of the second layer is about 1 mm, and the average diameter of the pores of the second layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the second layer is about 1 mm, and the average diameter of the pores of the second layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the second layer is about 1.5 mm, and the average diameter of the pores of the second layer is about 100 μm to about 1,000 μm. In some embodiments, the thickness of the second layer is about 1.5 mm, and the average diameter of the pores of the second layer is at least about 100 μm. In some embodiments, the thickness of the second layer is about 1.5 mm, and the average diameter of the pores of the second layer is at most about 1,000 μm. In some embodiments, the thickness of the second layer is about 1.5 mm, and the average diameter of the pores of the second layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the second layer is about 1.5 mm, and the average diameter of the pores of the second layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the second layer is about 2 mm, and the average diameter of the pores of the second layer is about 100 μm to about 1,000 μm. In some embodiments, the thickness of the second layer is about 2 mm, and the average diameter of the pores of the second layer is at least about 100 μm. In some embodiments, the thickness of the second layer is about 2 mm, and the average diameter of the pores of the second layer is at most about 1,000 μm. In some embodiments, the thickness of the second layer is about 2 mm, and the average diameter of the pores of the second layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the second layer is about 2 mm, and the average diameter of the pores of the second layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the second layer is about 1 mm, and the average diameter of the pores of the second layer is about 50 μm to about 355 μm. In some embodiments, the thickness of the second layer is about 1 mm, and the average diameter of the pores of the second layer is at least about 50 μm. In some embodiments, the thickness of the second layer is about 1 mm, and the average diameter of the pores of the second layer is at most about 355 μm. In some embodiments, the thickness of the second layer is about 1 mm, and the average diameter of the pores of the second layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the second layer is about 1 mm, and the average diameter of the pores of the second layer is about 50 μm, about 75 μm, about 100 μm, about 150 μm, about 200 μm, about 250 μm, about 300 μm, or about 355 μm.

In some embodiments, the thickness of the second layer is about 1.5 mm, and the average diameter of the pores of the second layer is about 50 μm to about 355 μm. In some embodiments, the thickness of the second layer is about 1.5 mm, and the average diameter of the pores of the second layer is at least about 50 μm. In some embodiments, the thickness of the second layer is about 1.5 mm, and the average diameter of the pores of the second layer is at most about 355 μm. In some embodiments, the thickness of the second layer is about 1.5 mm, and the average diameter of the pores of the second layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the second layer is about 1.5 mm, and the average diameter of the pores of the second layer is about 50 μm, about 75 μm, about 100 μm, about 150 μm, about 200 μm, about 250 μm, about 300 μm, or about 355 μm.

In some embodiments, the thickness of the second layer is about 2 mm, and the average diameter of the pores of the second layer is about 50 μm to about 355 μm. In some embodiments, the thickness of the second layer is about 2 mm, and the average diameter of the pores of the second layer is at least about 50 μm. In some embodiments, the thickness of the second layer is about 2 mm, and the average diameter of the pores of the second layer is at most about 355 μm. In some embodiments, the thickness of the second layer is about 2 mm, and the average diameter of the pores of the second layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the second layer is about 2 mm, and the average diameter of the pores of the second layer is about 50 μm, about 75 μm, about 100 μm, about 150 μm, about 200 μm, about 250 μm, about 300 μm, or about 355 μm.

In some cases, the polymer of the second layer may be nanoporous, wherein the average diameter of the pores ranges from 30 nm to 100 nm, such as, 30-75 nm, 30-50 nm, 40-100 nm, 40-75 nm, 40-50 nm, 50-100 nm, or 50-75 nm, e.g., 30 nm, 40 nm, 50 nm, 60 nm, or 80 nm.

In other cases, the polymer of the second layer may be microporous, wherein the average diameter of the pores ranges from 1 μm to 3 μm (e.g., 2 μm±0.5 μm diameter) with a connectivity diameter ranging from 900 nm-100 nm, such as, 900 nm-150 nm, 900 nm-200 nm, 700 nm-100 nm, 700 nm-150 nm, 700 nm-200 nm, 500 nm-100 nm, 500 nm-150 nm, 300 nm-100 nm, 300 nm-150 nm, or 300 nm-200 nm.

In some cases, the polymer of the second layer may have an average connectivity diameter ranging from about 10 nm to about 2 μm. For example, the plurality of pores present in the second layer may have an average connectivity diameter ranging from about 20 nm to about 300 nm, from about 30 nm to about 300 nm, from about 10 nm to about 200 nm, from about 20 nm to about 200 nm, from about 30 nm to about 200 nm, from about 10 nm to about 100 nm, from about 20 nm to about 100 nm, from about 30 nm to about 100 nm, from about 30 nm to about 50 nm, from about 30 nm to about 40 nm, from about 20 nm to about 250 nm, from about 20 nm to about 150 nm, from about 25 to about 150 nm, from about 200 nm to about 500 nm, from about 350 nm to about 500 nm, from about 500 nm to about 1.5 μm, or from about 1.0 μm to about 2.0 μm. In some cases, the plurality of pores in the second layer may have an average connectivity diameter of about 10 nm, about 20 nm, about 30 nm, about 40 nm, about 50 nm, about 60 nm, about 70 nm, about 80 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 550 nm, about 600 nm, about 650 nm, about 700 nm, about 750 nm, about 800 nm, about 850 nm, about 900 nm, about 950 nm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, or about 2.0 μm. In one aspect, the average connectivity diameter may be about 200 nm. In another aspect, the average connectivity diameter may be about 2.0 μm.

In some embodiments, the respirator face mask comprises a third layer as shown in FIG. 1A. The third layer further filters any pathogens and/or hazards that may have passed through the first layer, and the second layer if present. The third layer may comprise a nanoporous polymer. The nanoporous polymer may comprise methacrylate polymers, polyethylene-imine and dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapetite, poly(lactic acid), polyhydroxyvalerte and copolymers, polyhydroxybutyrate and copolymers, polycaprolactone, polydiaxanone, polyanhydrides, polycyanocrylates, poly(amino acids), poly(orthoesters), polyesters, collagen, gelatin, cellulose polymers, chitosans, alginates, polycaprolactone (PCL), polyvinylidene fluoride (PVDF), polytetrafluoroethylene (PTFE), polyethylene (PE), methacrylate polymer, polyethyleneimine, polyethyleneimine-dextran sulfate, poly(vinylsiloxane), laminin, Teflon, polyethylene terephthalate (PET), or a combination thereof. In some cases, the nanoporous polymer is poly(caprolactone)(PCL).

In some embodiments, the thickness of the third layer is about 1 mm to about 2 mm. In some embodiments, the thickness of the third layer is at least about 1 mm. In some embodiments, the thickness of the third layer is at most about 2 mm. In some embodiments, the thickness of the third layer is about 1 mm to about 1.5 mm, about 1 mm to about 2 mm, or about 1.5 mm to about 2 mm. In some embodiments, the thickness of the third layer is about 1 mm, about 1.5 mm, or about 2 mm.

In some embodiments, the average diameter of the pores of the third layer is about 50 μm to about 500 μm. In some embodiments, the average diameter of the pores of the third layer is at least about 50 μm. In some embodiments, the average diameter of the pores of the third layer is at most about 500 μm. In some embodiments, the average diameter of the pores of the third layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 75 μm to about 100 μm, about 75 μm to about 150 μm, about 75 μm to about 200 μm, about 75 μm to about 250 μm, about 75 μm to about 300 μm, about 75 μm to about 350 μm, about 75 μm to about 400 μm, about 75 μm to about 450 μm, about 75 μm to about 500 μm, about 100 μm to about 150 μm, about 100 μm to about 200 μm, about 100 μm to about 250 μm, about 100 μm to about 300 μm, about 100 μm to about 350 μm, about 100 μm to about 400 μm, about 100 μm to about 450 μm, about 100 μm to about 500 μm, about 150 μm to about 200 μm, about 150 μm to about 250 μm, about 150 μm to about 300 μm, about 150 μm to about 350 μm, about 150 μm to about 400 μm, about 150 μm to about 450 μm, about 150 μm to about 500 μm, about 200 μm to about 250 μm, about 200 μm to about 300 μm, about 200 μm to about 350 μm, about 200 μm to about 400 μm, about 200 μm to about 450 μm, about 200 μm to about 500 μm, about 250 μm to about 300 μm, about 250 μm to about 350 μm, about 250 μm to about 400 μm, about 250 μm to about 450 μm, about 250 μm to about 500 μm, about 300 μm to about 350 μm, about 300 μm to about 400 μm, about 300 μm to about 450 μm, about 300 μm to about 500 μm, about 350 μm to about 400 μm, about 350 μm to about 450 μm, about 350 μm to about 500 μm, about 400 μm to about 450 μm, about 400 μm to about 500 μm, or about 450 μm to about 500 μm. In some embodiments, the average diameter of the pores of the third layer is about 50 μm, about 75 μm, about 100 μm, about 150 μm, about 200 μm, about 250 μm, about 300 μm, about 350 μm, about 400 μm, about 450 μm, or about 500 μm.

In some embodiments, the average diameter of the pores of the third layer is about 500 μm to about 1,000 μm. In some embodiments, the average diameter of the pores of the third layer is at least about 500 μm. In some embodiments, the average diameter of the pores of the third layer is at most about 1,000 μm. In some embodiments, the average diameter of the pores of the third layer is about 500 μm to about 550 μm, about 500 μm to about 600 μm, about 500 μm to about 650 μm, about 500 μm to about 700 μm, about 500 μm to about 750 μm, about 500 μm to about 800 μm, about 500 μm to about 850 μm, about 500 μm to about 900 μm, about 500 μm to about 950 μm, about 500 μm to about 1,000 μm, about 550 μm to about 600 μm, about 550 μm to about 650 μm, about 550 μm to about 700 μm, about 550 μm to about 750 μm, about 550 μm to about 800 μm, about 550 μm to about 850 μm, about 550 μm to about 900 μm, about 550 μm to about 950 μm, about 550 μm to about 1,000 μm, about 600 μm to about 650 μm, about 600 μm to about 700 μm, about 600 μm to about 750 μm, about 600 μm to about 800 μm, about 600 μm to about 850 μm, about 600 μm to about 900 μm, about 600 μm to about 950 μm, about 600 μm to about 1,000 μm, about 650 μm to about 700 μm, about 650 μm to about 750 μm, about 650 μm to about 800 μm, about 650 μm to about 850 μm, about 650 μm to about 900 μm, about 650 μm to about 950 μm, about 650 μm to about 1,000 μm, about 700 μm to about 750 μm, about 700 μm to about 800 μm, about 700 μm to about 850 μm, about 700 μm to about 900 μm, about 700 μm to about 950 μm, about 700 μm to about 1,000 μm, about 750 μm to about 800 μm, about 750 μm to about 850 μm, about 750 μm to about 900 μm, about 750 μm to about 950 μm, about 750 μm to about 1,000 μm, about 800 μm to about 850 μm, about 800 μm to about 900 μm, about 800 μm to about 950 μm, about 800 μm to about 1,000 μm, about 850 μm to about 900 μm, about 850 μm to about 950 μm, about 850 μm to about 1,000 μm, about 900 μm to about 950 μm, about 900 μm to about 1,000 μm, or about 950 μm to about 1,000 μm. In some embodiments, the average diameter of the pores of the third layer is about 500 μm, about 550 μm, about 600 μm, about 650 μm, about 700 μm, about 750 μm, about 800 μm, about 850 μm, about 900 μm, about 950 μm, or about 1,000 μm.

In some embodiments, the thickness of the third layer is about 1 mm, and the average diameter of the pores of the third layer is about 100 μm to about 1,000 μm. In some embodiments, the thickness of the third layer is about 1 mm, and the average diameter of the pores of the third layer is at least about 100 μm. In some embodiments, the thickness of the third layer is about 1 mm, and the average diameter of the pores of the third layer is at most about 1,000 μm. In some embodiments, the thickness of the third layer is about 1 mm, and the average diameter of the pores of the third layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the third layer is about 1 mm, and the average diameter of the pores of the third layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the third layer is about 1.5 mm, and the average diameter of the pores of the third layer is about 100 μm to about 1,000 μm. In some embodiments, the thickness of the third layer is about 1.5 mm, and the average diameter of the pores of the third layer is at least about 100 μm. In some embodiments, the thickness of the third layer is about 1.5 mm, and the average diameter of the pores of the third layer is at most about 1,000 μm. In some embodiments, the thickness of the third layer is about 1.5 mm, and the average diameter of the pores of the third layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the third layer is about 1.5 mm, and the average diameter of the pores of the third layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the third layer is about 2 mm, and the average diameter of the pores of the third layer is about 100 μm to about 1,000 μm. In some embodiments, the thickness of the third layer is about 2 mm, and the average diameter of the pores of the third layer is at least about 100 μm. In some embodiments, the thickness of the third layer is about 2 mm, and the average diameter of the pores of the third layer is at most about 1,000 μm. In some embodiments, the thickness of the third layer is about 2 mm, and the average diameter of the pores of the third layer is about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. In some embodiments, the thickness of the third layer is about 2 mm, and the average diameter of the pores of the third layer is about 100 μm, about 200 μm, about 300 μm, about 400 μm, about 500 μm, about 600 μm, about 700 μm, about 800 μm, about 900 μm, or about 1,000 μm.

In some embodiments, the thickness of the third layer is about 1 mm, and the average diameter of the pores of the third layer is about 50 μm to about 355 μm. In some embodiments, the thickness of the third layer is about 1 mm, and the average diameter of the pores of the third layer is at least about 50 μm. In some embodiments, the thickness of the third layer is about 1 mm, and the average diameter of the pores of the third layer is at most about 355 μm. In some embodiments, the thickness of the third layer is about 1 mm, and the average diameter of the pores of the third layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the third layer is about 1 mm, and the average diameter of the pores of the third layer is about 50 μm, about 75 μm, about 100 μm, about 150 μm, about 200 μm, about 250 μm, about 300 μm, or about 355 μm.

In some embodiments, the thickness of the third layer is about 1.5 mm, and the average diameter of the pores of the third layer is about 50 μm to about 355 μm. In some embodiments, the thickness of the third layer is about 1.5 mm, and the average diameter of the pores of the third layer is at least about 50 μm. In some embodiments, the thickness of the third layer is about 1.5 mm, and the average diameter of the pores of the third layer is at most about 355 μm. In some embodiments, the thickness of the third layer is about 1.5 mm, and the average diameter of the pores of the third layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the third layer is about 1.5 mm, and the average diameter of the pores of the third layer is about 50 μm, about 75 μm, about 100 μm, about 150 μm, about 200 μm, about 250 μm, about 300 μm, or about 355 μm.

In some embodiments, the thickness of the third layer is about 2 mm, and the average diameter of the pores of the third layer is about 50 μm to about 355 μm. In some embodiments, the thickness of the third layer is about 2 mm, and the average diameter of the pores of the third layer is at least about 50 μm. In some embodiments, the thickness of the third layer is about 2 mm, and the average diameter of the pores of the third layer is at most about 355 μm. In some embodiments, the thickness of the third layer is about 2 mm, and the average diameter of the pores of the third layer is about 50 μm to about 75 μm, about 50 μm to about 100 μm, about 50 μm to about 150 μm, about 50 μm to about 200 μm, about 50 μm to about 250 μm, about 50 μm to about 300 μm, about 50 μm to about 355 μm, about 75 μm to about 100 μm, about 75 μm to about 150 μm, about 75 μm to about 200 μm, about 75 μm to about 250 μm, about 75 μm to about 300 μm, about 75 μm to about 355 μm, about 100 μm to about 150 μm, about 100 μm to about 200 μm, about 100 μm to about 250 μm, about 100 μm to about 300 μm, about 100 μm to about 355 μm, about 150 μm to about 200 μm, about 150 μm to about 250 μm, about 150 μm to about 300 μm, about 150 μm to about 355 μm, about 200 μm to about 250 μm, about 200 μm to about 300 μm, about 200 μm to about 355 μm, about 250 μm to about 300 μm, about 250 μm to about 355 μm, or about 300 μm to about 355 μm. In some embodiments, the thickness of the third layer is about 2 mm, and the average diameter of the pores of the third layer is about 50 μm, about 75 μm, about 100 μm, about 150 μm, about 200 μm, about 250 μm, about 300 μm, or about 355 μm.

In some cases, the average diameter of the pores of the nanoporous polymer ranges from about 50 nm to about 300 nm. In some cases, the average diameter of the pores is about 50 nm to 100 nm, about 75 nm to about 125 nm, about 100 nm to about 150 nm, about 125 nm to about 175 nm, about 150 nm to about 200 nm, about 175 nm to about 225 nm, about 200 nm to about 250 nm, about 225 nm to about 275 nm, or about 250 nm to about 300 nm.

In some cases, the connectivity diameter of the nanoporous polymer of the third layer ranges from about 100 nm to about 900 nm, about 150 nm to about 900 nm, about 200 nm to about 900 nm, about 100 nm to about 700 nm, about 150 nm to about 700 nm, about 200 nm to about 700 nm, about 100 nm to about 500 nm, about 150 nm to about 500 nm, about 100 nm to about 300 nm, about 150 nm to about 300 nm, or about 200 nm to about 300 nm.

The third layer can be positively charged or negatively charge to bind to airborne microorganisms such as bacteria, fungi and virus. The third layer can comprise one or more metal ions, one or more electrophiles, one or more nucleophiles, or a combination thereof. The one or more metal ions may be copper, zinc, or a combination thereof.

The third layer of the respirator face mask may be woven. The third layer may be spun or electrospun. The third layer may be cast. The third layer may be blown. The third layer may be molded. The third layer may be etched. In some embodiments, the third layer may be fabricated by extrusion, casting, or molding. In some embodiments, the third layer may be die cut, sewn, molded, injection molded, cast, stamped, printed, or extruded.

The respirator face mask may optionally comprise a fourth layer as shown in FIG. 1A. The fourth layer can provide additional comfort as the layer in contact with the skin of the subject wearing the mask. The fourth layer may comprise natural fibers, natural-like fibers, or a combination thereof. The fourth layer may include condensation wicking and/or absorbent to provide comfort.

In some cases, the filtration media may comprise a single layer, as shown in FIG. 1B. Alternatively, the filtration media may comprise a first layer and a second layer, as illustrated in FIG. 1C. In some embodiments, the filtration media may comprise a first layer, a second layer, and a third layer, as shown in FIG. 1D. The multiple layers of the filtration media may comprise similar properties. In some cases, the multiple layers of the filtration media may comprise different properties. A filtration media comprising a single layer may exhibit different performance characteristics (e.g., filtration efficiency and/or breathability) compared to a filtration media comprise a plurality of layers.

In any of the embodiments described herein, the layers of the filtration media may comprise a plurality of pores. The plurality of pores may be disposed on a portion of the one or more layers of the filtration media. The plurality of pores may have a pore density that permits high filtration efficiency. In some cases, the pore density may be at least about 10 pores/mm², at least about 20 pores/mm², at least about 30 pores/mm², at least about 40 pores/mm², at least about 50 pores/mm², at least about 60 pores/mm², at least about 70 pores/mm², at least about 80 pores/mm², at least about 90 pores/mm², at least about 100 pores/mm², at least about 200 pores/mm², at least about 300 pores/mm², at least about 400 pores/mm², at least about 500 pores/mm², at least about 600 pores/mm², at least about 700 pores/mm², at least about 800 pores/mm², at least about 900 pores/mm², at least about 1000 pores/mm², at least about 2000 pores/mm², at least about 3000 pores/mm², at least about 4000 pores/mm², at least about 5000 pores/mm², at least about 6000 pores/mm², at least about 7000 pores/mm², at least about 8000 pores/mm², at least about 9000 pores/mm², at least about 10,000 pores/mm², at least about 50,000 pores/mm², at least about 100,000 pores/mm², at least about 500,000 pores/mm², at least about 1,000,000 pores/mm², at least about 5,000,000 pores/mm², at least about 10,000,000 pores/mm², at least about 50,000,000 pores/mm², at least about 100,000,000 pores/mm², or more. In some cases, the density of the membrane or the one or more layers of the membrane having the plurality of pores may be from about 0.01 milligrams per cubic millimeter (mg/mm³) to about 1 mg/mm³. In some cases, the density of the membrane or the one or more layers of the membrane having the plurality of pores may be from about 0.1342+/−0.2 mg/mm³ to about 0.1519+/−0.2 mg/mm³. In some cases, the density of the membrane or the one or more layers of the membrane having the plurality of pores may be from about 0.1342+/−0.5 mg/mm³ to about 0.1519+/−0.5 mg/mm³.

Multiple Layers

In some embodiments, the filtration device (e.g., a respirator face mask) comprises a first layer and a second layer as described herein. In some embodiments, the respirator face mask may comprise a first layer, a second layer, and a third layer as described herein. In other embodiments, the respirator face mask may comprise a first layer, a second layer, a third layer, and/or a fourth layer as described herein. In another embodiment, the respirator face mask comprises a first layer, a second layer, a third layer and/or a fourth layer as described herein. The first layer may be different than the second layer, the third layer, and/or the fourth layer. The second layer may be different than the first layer, the third layer, and/or the fourth layer. The third layer may be different than the first layer, the second layer, and/or the fourth layer. The fourth layer may be different than the first layer, the second layer, and/or the third layer. In some cases, the first layer may be the same as or similar to the second layer, the third layer, and/or the fourth layer. In some cases, the second layer may be the same as or similar to the first layer, the third layer, and/or the fourth layer. In some cases, the third layer may be the same as or similar to the first layer, the second layer, and/or the fourth layer. In some cases, the fourth layer may be the same as or similar to the first layer, the second layer, and/or the third layer. In some embodiments, at least two layers of the first layer, the second layer, the third layer, and the fourth layer may be the same or similar (e.g., in material composition, performance, and/or functionality). In other embodiments, at least two layers of the first layer, the second layer, the third layer, and the fourth layer may be different (e.g., in material composition, performance, and/or functionality).

The layers of the respirator face masks or filtration media described herein may be manufactured using different molding techniques. Each layer may be blown, molded, etched, die cut, sewn, injected, cast, stamped, printed, or a combination thereof. In some cases, one or more layers of the respirator face masks or filtration media described herein may be woven, spun, electrospun, blown, molded, or etched. In some embodiments, the one or more layers may be fabricated by extrusion, casting, or molding. In some embodiments, one or more layers of the respirator face masks or filtration media described herein may be die cut, sewn, molded, injection molded, cast, stamped, printed, or extruded.

In some cases, the respirator face masks provided herein may be for single use. In other cases, the respirator face masks described herein are reusable. For reusable respirator face masks, the respirator face mask may be sanitized by washing, ethylene oxide sterilization, exposure to electromagnetic radiation, irradiation, exposure to high strength magnetic fields, a disinfectant, or a combination thereof. In some cases, the electromagnetic radiation is ultraviolet radiation.

In some cases, the filtration media and/or the one or more layers or membranes of the filtration media may be biodegradable as described elsewhere herein. The biodegradability of the filtration media may be associated with a chemical structure of the layers or membranes of the filtration media.

In some cases, the one or more layers or membranes of the filtration media may comprise a crystal structure that enhances the reusability of the filtration media and the one or more layers or membranes of the filtration media. In some cases, the crystal structure may permit the one or more layers or membranes to be melted and remade, refurbished, refashioned, reshaped, and/or reconditioned (e.g., for reuse or for use in another filtration device). For example, after a first use of the filtration media to filter one or more particles, the filtration media may be heated or melted. After being heated or melted, the filtration media may be remade into another filtration media for subsequent use in the same filtration device, or in another filtration device. Alternatively, after being heated or melted, the filtration media may be refurbished or reconditioned for reuse by the same user, or for subsequent use by another user. In some cases, after being heated or melted, the filtration media may be refashioned to filter different types of particles. In other cases, after being heated or melted, the filtration media may be reshaped to be compatible with different users or different systems that utilize or require particle filtration.

Performance Characteristics

The respirator face masks and filtration media described herein may exhibit certain desirable performance characteristics that provide a high filtration efficiency and low air flow resistance or delta P. As used herein, the term “delta P” may refer to a delta or change in pressure between a first region and a second region. The first region may correspond to a region external to the mask. The second region may correspond to a region that is between the mask and a user or subject wearing the mask. The second region may be adjacent to or in fluid communication with an air flow path that extends into a respiratory tract of the user or subject. The respiratory tract may be a bodily orifice through which the user or subject can breathe (e.g., a mouth or a nostril). As described elsewhere herein, the respirator face mask may comprise one or more layers comprising one or more polycaprolactone membranes.

The filtration media and the filtration devices disclosed herein may exhibit certain desirable performance characteristics. The performance characteristics may comprise fluid resistance, bacterial filtration efficiency, particulate filtration efficiency, and differential pressure (or delta P).

The performance characteristics may comprise, for example, fluid resistance, which represents the ability of a filtration media or material to minimize or reduce fluids from traveling through the filtration media or material. The fluid resistance may be determined based on one or more industry test standards (e.g., ASTM F1862). The filtration media of the present disclosure may exhibit a fluid resistance that prevents one or more fluids (e.g., synthetic blood) having a certain threshold stream velocity from penetrating through the filtration media. The threshold stream velocity may correspond to a velocity of a fluid that is pressurized to at least about 80 mmHg, at least about 120 mmHg, at least about 160 mmHg, or more.

The performance characteristics may comprise, for example, Bacterial Filtration Efficiency (BFE), which represents the percentage of aerosol particulates that are filtered. BFE may be used as a measure of the efficiency of the filtration media in filtering bacteria passing through the filtration media. BFE may be determined based on one or more industry test standards (e.g., ASTM F2101). The aerosol particulates may comprise, for example, a sample droplet comprising Staphylococcus aureus with an average particle size of about 0.6 microns to about 0.8 microns. The filtration media of the present disclosure may exhibit a BFE of at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or more.

The performance characteristics may comprise, for example, sub-micron Particulate Filtration Efficiency (PFE), which represents the percentage of submicron particulates filtered at a threshold size (e.g., 0.1 microns). PFE may be used as a measure of the efficiency of the filtration media in filtering particles passing through it. PFE may be determined based on one or more industry test standards (e.g., ASTM F2299). The submicron particulates used to determine PFE may comprise one or more aerosolized particles. The filtration media of the present disclosure may exhibit a PFE of at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or more.

The performance characteristics may comprise, for example, differential pressure (delta P), which represents the pressure drop across the filtration media. The differential pressure may correspond to a resistance to air flow, and may be measured in mmH₂O/cm². Differential pressure may be determined based on one or more industry test standards (e.g., ASTM F2100). The filtration media of the present disclosure may exhibit a delta P of at most about 5 mmH₂O/cm², at most about 4 mmH₂O/cm², at most about 3 mmH₂O/cm², at most about 2 mmH₂O/cm², at most about 1 mmH₂O/cm², at most about 0.9 mmH₂O/cm², at most about 0.8 mmH₂O/cm², at most about 0.7 mmH₂O/cm², at most about 0.6 mmH₂O/cm², at most about 0.5 mmH₂O/cm², at most about 0.4 mmH₂O/cm², at most about 0.3 mmH₂O/cm², at most about 0.2 mmH₂O/cm², at most about 0.1 mmH₂O/cm², or less.

### Examples

The performance characteristics of the respirator face masks comprising the membranes described herein were verified using various tests. These various tests are described in detail below.

A first testing procedure was performed to evaluate the non-viable particle filtration efficiency (PFE) of the membranes (also referred to in this section as “test article”). Monodispersed polystyrene latex spheres (PSL) were nebulized (atomized), dried, and passed through the test article at an air flow rate. The particles that passed through the test article were enumerated using a laser particle counter.

A one-minute count was performed, with the test article in the testing setup/system. A one-minute control count was performed, without a test article in the testing setup/system, before and after the performance testing for each test article. Control counts were performed to determine the average number of particles delivered to the test article. The filtration efficiency was calculated using the number of particles penetrating the test article compared to the average of the control values.

During testing and controls, the air flow rate was maintained at 1 cubic foot of air per minute (CFM)±5%. The testing procedure employed the basic particle filtration method described in ASTM F2299, with some exceptions. Notably, the instant testing procedure incorporated a non-neutralized challenge. In real use, particles carry a charge, thus this challenge represents a more natural state. The non-neutralized aerosol is also specified in the FDA guidance document on surgical face masks. All test method acceptance criteria were met. Testing was performed in compliance with U.S. Food and Drug Administration good manufacturing practice (GMP) regulations 21 C.F.R. Parts 210, 211 and 820.

In one instance, the testing procedure was conducted using the following parameters:

Test Side: Either Side

Area Tested: 91.5 cm²

Particle Size: 0.3 μm

Laboratory Conditions: 22.1° Celsius, 21% relative humidity (RH); 21.8° C., 22% RH.

The testing procedure yielded the results shown in Table 1 below for a single layer membrane. The minimum filtration efficiency was 66% with a test article count of 3,648+/−2,000 and an average control count of 10,593+/−2,000. The maximum filtration efficiency was 76% with a test article count of 2,504+/−2,000 and an average control count of 10,448+/−2,000.

TABLE 1
Test results for single layer membrane

| Single | Test Article | Average Control | Filtration |
|---|---|---|---|
| Layer | Counts | Counts | Efficiency |
| Min | 3,648 +/− 2,000 | 10,593 +/− 2,000 | 66% |
| Max | 2,504 +/− 2,000 | 10,448 +/− 2,000 | 76% |

The testing procedure yielded the results shown in Table 2 below for a double layer membrane. The minimum filtration efficiency was 77% with a test article count of 2,282+/−2,000 and an average control count of 10,074+/−2,000. The maximum filtration efficiency was about 94% with a test article count of about 646+/−2,000 and an average control count of about 10,606+/−2,000.

TABLE 2
Test results for double layer membrane

| Double | Test Article | Average Control | Filtration |
|---|---|---|---|
| Layer | Counts | Counts | Efficiency |
| Min | 2,282 +/− 2,000 | 10,074 +/− 2,000 | 77% |
| Max | 646 +/− 2,000 | 10,606 +/− 2,000 | 94% |

A second testing procedure was performed to evaluate the delta P performance of the membranes (also referred to herein as “test article(s)”). The Delta P test was performed to determine the breathability of test articles by measuring the differential air pressure on either side of the test article using a manometer, at a constant flow rate. The Delta P test complies with EN 14683:2019, Annex C and ASTM F2100-19. All test method acceptance criteria were met. Testing was performed in compliance with U.S. Food and Drug Administration good manufacturing practice (GMP) regulations 21 CFR Parts 210, 211 and 820. It is noted herein that breathability is improved when there is a lower Delta P. As shown below, both single and double layer masks met the requirements and performance criteria for a passing article.

In one instance, the testing procedure was conducted using the following parameters:

Test Side: Either

Delta P Flow Rate: 8 Liters per minute (L/min)

Conditioning Parameters: 85±5% relative humidity (RH) and 21±5° Celsius for a minimum of 4 hours.

The testing procedure yielded the results shown in Table 3 below for a single layer membrane. The minimum delta P for a single layer was about 0.2 mm H₂O/cm² or 1.9 pascals per square centimeter (Pa/cm²). The maximum delta P for a single layer was about 0.3 mm H₂O/cm² or 3.4 Pa/cm².

TABLE 3
Delta P test results for single layer membrane

| Single | Delta P | Delta P |
|---|---|---|
| Layer | (mm H₂O/cm²) | (Pa/cm²) |
| Min | 0.2 | 1.9 |
| Max | 0.3 | 3.4 |

The testing procedure yielded the results shown in Table 4 below for a double layer membrane. The minimum delta P for a double layer was about 0.4 mm H₂O/cm² or 3.7 pascals per square centimeter (Pa/cm²). The maximum delta P for a double layer was about 4.6 mm H₂O/cm² or 45.1 Pa/cm².

TABLE 4
Delta P test results for double layer membrane

| Double | Delta P | Delta P |
|---|---|---|
| Layer | (mm H₂O/cm²) | (Pa/cm²) |
| Min | 0.4 | 3.7 |
| Max | 4.6 | 45.1 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing embodiments of the disclosure. It is intended that the following claims define the scope of the disclosure and that methods and structures within the scope of these claims and their equivalents be covered thereby.

What is claimed is:

1. A filtration media comprising:
a membrane comprising one or more polymeric layers configured to filter one or more particles, wherein the membrane comprises a plurality of pores having an average diameter of at most about 175 μm (micrometers),
wherein the membrane provides a filtration efficiency of at least about 65%, and
wherein the membrane exhibits a delta P of at most about 5 mm H₂O/cm².

2. The filtration media of claim 1, wherein the polymeric layer comprises a polymer selected from the group consisting of methacrylate polymer, polyethylene-imine, dextran sulfate, poly(vinylsiloxane) ecopolymerepolyethyleneimine, phosphorylcholine, poly(ethyl methacrylate), polyurethane, poly(ethylene glycol), poly(lactic-glycolic acid), hydroxyapetite, poly(lactic acid), polyhydroxyvalerte, polyhydroxybutyrate, polycaprolactone, polydiaxanone, polyanhydride, polycyanocrylate, poly(amino acid), poly(orthoester), polyester, collagen, gelatin, cellulose polymers, chitosan, alginate, polypropylene, polystyrene, Teflon, and polyethylene terephthalate (PET).

3. The filtration media of claim 1 or 2, wherein at least one of the one or more polymeric layers comprises an active agent configured to devitalize or deactivate one or more microorganisms or toxins.

4. The filtration media of any one of claims 1-3, wherein at least one of the one or more polymeric layers is positively or negatively charged to bind to the one or more particles.

5. The filtration media of any one of claims 1-4, wherein at least one of the one or more polymeric layers is acidic.

6. The filtration media of any one of claims 1-5, wherein the average diameter of the plurality of pores is from about 125 μm (micrometers) to about 175 μm.

7. The filtration media of any one of claims 1-6, wherein the plurality of pores have an average connectivity diameter ranging from about 10 nanometers (nm) to about 200 micrometers (μm).

8. The filtration media of any one of claims 1-7, wherein the one or more polymeric layers comprise a first polymeric layer with a first set of properties and a second polymeric layer with a second set of properties.

9. The filtration media of claim 8, wherein the first and second set of properties comprise a polymeric layer thickness, a pore size, a pore connectivity diameter, a pore density, or any combination thereof.

10. The filtration media of claim 8, wherein the first and second set of properties are the same.

11. The filtration media of claim 8, wherein the first and second set of properties are different.

12. The filtration media of any one of claims 1-11, wherein at least one of the one or more polymeric layers is woven, spun, electrospun, blown, etched, die cut, sewn, molded, injection molded, cast, stamped, printed, or extruded.

13. The filtration media of any one of claims 1-12, wherein at least one of the one or more polymeric layers comprises a film or a scaffold.

14. The filtration media of any one of claims 1-13, wherein the membrane comprises a single-use membrane.

15. The filtration media of any one of claims 1-13, wherein the membrane comprises a reusable membrane.

16. The filtration media of any one of claims 1-15, wherein the membrane has a filtration efficiency of at least about 70%.

17. The filtration media of any one of claims 1-15, wherein the membrane has a filtration efficiency of at least about 80%.

18. The filtration media of any one of claims 1-15, wherein the membrane has a filtration efficiency of at least about 90%.

19. The filtration media of any one of claims 1-18, wherein the delta P of the membrane is from about 0.2 mm H₂O/cm² to 5 mm H₂O/cm².

20. The filtration media of any one of claims 1-19, wherein the one or more particles have a particle size that is at least about 100 nm.

21. The filtration media of any one of claims 1-20, wherein the membrane is biodegradable.

22. The filtration media of any one of claims 1-20, wherein the membrane is capable of being remade, refurbished, refashioned, reshaped, or reconditioned after at least a portion of the membrane is heated or melted.

23. A device for filtering one or more particles, comprising:
the filtration media of claim 1; and
at least one securement member for securing the filtration media to a subject or an object.

24. The device of claim 23, further comprising an additional layer comprising a condensation wicking or absorbent material.

25. The device of claim 24, wherein the additional layer comprises one or more natural fibers or natural-like fibers.

26. The device of any one of claims 23-25, wherein the device comprises a component configured to receive the filtration media.

27. The device of any one of claims 23-26, wherein the filtration media is swappable or switchable after one or more uses.

28. The device of any one of claims 23-27, wherein the device comprises a respirator face mask.

29. A mask for filtering one or more airborne particles, comprising:
a membrane comprising one or more polymeric layers configured to filter one or more particles, wherein the one or more polymeric layers comprise polycaprolactone, wherein the membrane comprises a plurality of pores; and
at least one securement member for securing the membrane to a subject or an object.

30. The mask of claim 29, wherein the plurality of pores have an average diameter that is from about 125 μm (micrometers) to about 175 μm.

31. The mask of claim 29 or 30, wherein the mask has a filtration efficiency of at least about 90%.

32. The mask of any one of claims 29-31, wherein the mask exhibits a delta P that is from about 0.2 mm H₂O/cm² to about 5 mm H₂O/cm².

33. The filtration media of any one of claims 1-22, wherein the membrane comprising the plurality of pores has a density that is from about 0.1 mg/mm³ to about 0.2 mg/mm³.

34. The filtration media of any one of claims 1-22, wherein the membrane comprising the plurality of pores has a density that is from about 0.01 mg/mm³ to about 1 mg/mm³.

35. The filtration media of any one of claims 1-22, wherein the membrane comprising the plurality of pores has a pore density that is from about 10,000 pores/mm² to about 100,000 pores/mm².

36. The filtration media of any one of claims 1-22, wherein the membrane comprising the plurality of pores has a pore density of at least about 100,000 pores/mm².
